(19) United States
(12) Patent Application Publication
(10) Pub. No.: US 2017/0252394 A1
(43) Pub. Date: Sep. 7, 2017

(54) NATUROPATHIC WOUND TREATMENT AND ASSOCIATED METHODS

(71) Applicant: Renee Lynch, Payson, AZ (US)

(72) Inventor: Renee Lynch, Payson, AZ (US)

(73) Assignee: Kaloka Kare LLC, Payson, AZ (US)

(21) Appl. No.: 15/445,391

(22) Filed: Feb. 28, 2017

Related U.S. Application Data
(60) Provisional application No. 62/303,554, filed on Mar. 4, 2016.

(51) Int. Cl. A61K 36/889; A61K 36/886; A61K 9/06; A61K 33/38; A61K 9/00; A61K 35/644; A61K 31/355; A61K 31/716

(52) U.S. Cl. CPC A61K 36/889; A61K 31/355; A61K 36/886; A61K 31/716; A61K 33/38; A61K 9/0014; A61K 9/0019; A61K 35/644; A61K 9/06

(57) ABSTRACT

A naturopathic wound treatment product comprising a mixture of 0.55-0.75% by weight vitamin E oil, 40-60% by weight extra virgin coconut oil, 40-60% by weight aloe, and 0.1-0.3% by weight beta glucan, 3.5-4.5% by weight colloidal silver. All weights are based on a total weight of the product. The vitamin E oil, extra virgin coconut oil, aloe, beta glucan, and colloidal silver are combined to form a single mixture.

This application claims the benefit under 35 U.S.C. §119(e) of U.S. Provisional Patent Application Ser. No. 62/303,554 filed on Mar. 4, 2016 and titled Naturopathic Wound Treatment and Associated Methods, the entire content of which is incorporated herein by reference.

### FIELD OF THE INVENTION

The present invention relates to naturopathic wound treatment products and methods for manufacture. Specifically, the present invention is directed to a product that can be used to treat bed sores as well as other types of wounds.

### BACKGROUND

Modern treatment of skin wounds has included a barrage of synthetic ointments and therapies that not only stymie healing, but in some cases, inflame tissue causing further pain of the treated site. These products have included ingredients such as alcohol, dyes, perfumes, parabens, sulfates, animal derived ingredients, glutens, and GMOs. These very ingredients are also the cause of many skin related allergies that cause adverse skin reactions to otherwise healthy skin.

Therefore, there exists a need in the art for a natural wound treatment product that stimulates collagen production and promotes the regeneration of healthy granulation tissue in skin wounds. There exists a need for a product that uses all natural, organic ingredients including herbs and vitamins to facilitate the body's natural healing process. Such a product would hasten a patient's healing time, and in some cases, shorten hospital stays, thereby driving down associated healthcare costs.

The present invention uses all natural, organic ingredients and has proven to heal chronic skin wounds faster than synthetic treatments. It has been effective at relieving pain and has not shown the propensity towards allergic reactions that synthetic treatments may cause.

### SUMMARY OF THE INVENTION

With the above in mind, embodiments of the present invention are related to a naturopathic wound treatment product, which may include a mixture of 0.55-0.75% by weight vitamin E oil, 40-60% by weight extra virgin coconut oil, 40-60% by weight aloe, 0.1-0.3% by weight beta glucan, and 3.5-4.5% by weight colloidal silver. All weights are based on a total weight of the product. The vitamin E oil, extra virgin coconut oil, aloe, beta glucan, and colloidal silver may be combined to form a single mixture.

The aloe may be an aloe extract.

The mixture may be configured to be applied onto the skin of at least one of a human and an animal.

The mixture may be configured as an injectable fluid.

The naturopathic wound treatment product may include a mixture of 4.0-9.5% by weight vitamin E oil, 60.0-79.0% by weight extra virgin coconut oil, 1.3-6.0% by weight freeze dried aloe powder, 0.25-0.90% by weight beta glucan, and 0.25-4.50% by weight colloidal silver. AD weights may be based on a total weight of the product. The vitamin E oil, extra virgin coconut oil, freeze dried aloe powder, beta glucan, and colloidal silver may be combined to form a single mixture.

The product may include 9.8-18.0% by weight beeswax. The beeswax may be combined with the single mixture.

The vitamin E oil may be 4.63-9.03% by weight, the extra virgin coconut oil may be 61.06-77.06% by weight, the freeze dried aloe powder may be 2.48-5.88% by weight, the beta glucan may be 0.32-0.92% by weight, the colloidal silver may be 0.44-3.44% by weight, and the beeswax may be 10.27-17.77% by weight.

The vitamin E oil may be 5.63-8.93% by weight, the extra virgin coconut oil may be 63.06-75.06% by weight, the freeze dried aloe powder may be 2.58-4.88% by weight, the beta glucan may be 0.42-0.87% by weight, the colloidal silver may be 0.69-2.94% by weight, and the beeswax may be 12.27-17.67% by weight.

The vitamin E oil may be 6.63-8.83% by weight, the extra virgin coconut oil may be 65.06-73.06% by weight, the freeze dried aloe powder may be 2.68-3.88% by weight, the beta glucan may be 0.52-0.82% by weight, the colloidal silver may be 0.94-2.44% by weight, and the beeswax may be 14.27-17.57% by weight.

The vitamin E oil may be 7.63-8.73% by weight, the extra virgin coconut oil may be 67.06-71.06% by weight, the freeze dried aloe powder may be 2.78-3.38% by weight, the beta glucan may be 0.62-0.77% by weight, the colloidal silver may be 1.19-1.94% by weight, and the beeswax may be 16.27-17.47% by weight.

The vitamin E oil may be 8.63% by weight, the extra virgin coconut oil may be 69.06% by weight, the freeze dried aloe powder may be 2.88% by weight, the beta glucan may be 0.72% by weight, the colloidal silver may be 1.44% by weight, and the beeswax may be 17.27% by weight.

The mixture may be configured as a topical ointment.

The mixture may be configured as an injectable fluid.

The beeswax may be 100% organic and natural.

A method of manufacturing the naturopathic wound treatment product may include the steps of obtaining a portion of vitamin E oil, obtaining a portion of extra virgin coconut oil, obtaining a portion of freeze dried aloe powder, obtaining a portion of beta glucan, obtaining a portion of colloidal silver, obtaining a portion of beeswax, combining the portion of extra virgin coconut oil and the portion of beeswax in a bowl to form a combination coconut oil beeswax mixture, heating the combination coconut oil mixture to a liquid state to form a liquid combination coconut oil mixture, combining the portion of vitamin E oil with the liquid combination coconut oil mixture to from a combination vitamin E oil mixture, combining the portion of colloidal silver with the combination vitamin E oil mixture to form a combination colloidal silver mixture, combining the portion of beta glucan with the combination colloidal silver mixture to form a combination beta glucan mixture, combining the portion of freeze dried aloe with the combination beta glucan mixture to form a combination mixture.

The method of manufacture may include the step of stirring the combination vitamin E oil mixture for more than one minute.

The method of manufacture may include the step of stirring the combination colloidal silver mixture for more than one minute.

The method of manufacture may include the step of stirring the combination beta glucan mixture for more than one minute.

The method of manufacture may include the step of stirring the combination mixture for more than five minutes.

### BRIEF DESCRIPTION OF THE DRAWINGS

FIG. 1 is a flowchart for a method of manufacturing a naturopathic wound treatment product.

### DETAILED DESCRIPTION OF THE INVENTION

The present invention will now be described more fully hereinafter in which preferred embodiments of the invention are described. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

The present invention advantageously provides a naturopathic wound treatment composition that may include various mixtures of extra virgin coconut oil, aloe, colloidal silver, vitamin E, beta glucan, and beeswax.

Colloidal silver may be obtained from a variety of sources well known to those skilled in the art. For purposes of the present invention, the colloidal silver product is a solution of 250 parts per million colloidal silver suspended in pharmaceutical grade water. Accordingly, the colloidal silver suspended in pharmaceutical grade water is referred to hereinafter as colloidal silver. However, those skilled in the art will appreciate that the concentration of colloidal silver suspended in pharmaceutical grade water may range from 250 parts per million to 500 parts per million depending on the total percentage used in the composition. The colloidal silver and water combination included in the present invention may consist of between about 3.5% to about 4.5% by weight, more particularly from about 3.7% to about 4.2% by weight, and more particularly from about 3.8% to about 4.0% by weight. In one embodiment, the colloidal silver and water combination may consist of between about 0.25 to 4.50% by weight, more particularly between about 0.44 to 3.44% by weight, more particularly between about 0.69 to 2.94% by weight, more particularly between about 0.94 to 2.44% by weight, more particularly between about 1.19 to 1.94% by weight, and more particularly 1.44% by weight.

Extra virgin coconut oil may be obtained from a variety of sources well known to those skilled in the art. When present in the composition, extra virgin coconut oil may be present in an amount of about 40% to about 60% by weight, more particularly from about 45% to about 55% by weight, and more particularly from about 46% to about 48% by weight. In one embodiment, extra virgin coconut oil in the mixture may be present in the amount between about 60.00 to 79.00% by weight, more particularly between about 61.06 to 77.06% by weight, more particularly between about 63.06 to 75.06% by weight, more particularly between about 65.06 to 73.06% by weight, more particularly between about 67.06 to 71.06% by weight, and more particularly about 69.06% by weight.

Aloe is a naturally occurring product that may be obtained from a variety of sources well known to those skilled in the art. One embodiment may utilize aloe taken directly from its plant form. The aloe may be unprocessed or minimally processed and referred to as aloe extract. When aloe extract is present in the composition, the aloe is generally present in an amount of about 40% to about 60% by weight, more particularly from about 45% to about 55% by weight, and more particularly from about 46% to about 48% by weight. In one embodiment, freeze dried aloe powder may be utilized. In such an embodiment, the aloe may be present in a smaller amount by weight, due to the removal of the water from the aloe product during the freeze drying process. In embodiments utilizing freeze dried aloe powder, the freeze dried aloe powder may be present in an amount of about 1.3 to 6.0% by weight, more particularly from about 2.48-5.88% by weight, more particularly from about 2.58 to 4.88% by weight, more particularly from about 2.68 to 3.88% by weight, more particularly 2.78 to 3.38% by weight, and more particularly about 2.88% by weight.

Beta-1 3-D glucan (hereinafter “beta glucan”) may be obtained from a variety of sources well known to those skilled in the art. When present in the composition, beta glucan may be present in an amount of about 0.1% to about 0.3% by weight, more particularly from about 0.15% to about 0.25% by weight, and more particularly from about 0.19% to about 0.21% by weight. In one embodiment, beta glucan may be present in the mixture in the amount of between about 0.25 to 0.90% by weight, more particularly between about 0.32 to 0.92% by weight, more particularly between about 0.42 to 0.87% by weight, more particularly between 0.52 to 0.82% by weight, more particularly between about 0.62 to 0.77% by weight, and more particularly about 0.72% by weight.

Vitamin E oil may be obtained from a variety of sources well known to those skilled in the art. When present in the composition, Vitamin E oil may be present in an amount of about 0.55% to about 0.75% by weight, more particularly from about 0.6% to about 0.7% by weight, and more particularly from about 0.66% to about 0.68% by weight. In one embodiment Vitamin E may be present in an amount between about 4.00 to 9.50% by weight, more particularly between about 4.63 to 9.03% by weight, more particularly between about 5.63 to 8.93% by weight, more particularly between about 6.63 to 8.83% by weight, more particularly between about 7.63 to 8.73% by weight, more particularly about 8.63% by weight.

The composition of the present invention is used as a naturopathic wound treatment for expediting the recovery from bed sores and skin lesions. To reduce the possibility of drying, irritation, or other adverse skin reactions, synthetics such as alcohol, dyes; sulfates, perfumes; parabens, glutens, animal products and by-products have been intentionally avoided. The use of plant and mineral based ingredients advantageously meets the demand from the general public to use more natural and organic products versus products containing chemical or synthetic ingredients.

The composition of the present invention advantageously reduces the number and amount of chemicals being applied to the skin as compared to other skin care products known in the art. Additionally, the composition according to the present invention may advantageously be produced at a relatively low cost. The composition according to the present invention may also advantageously provide ecological advantages being that it may be a single-pack, non-pressurized product with varied application.

Thus, in some embodiments, the composition may consist essentially of vitamin E oil, extra virgin coconut oil, aloe, beta glucan, and colloidal silver, wherein the colloidal silver is a solution of 250 parts per million colloidal silver suspended in pharmaceutical grade water and the aloe may be an aloe extract. Amounts listed in the exemplary formulas demonstrating percentages are expressed by weight based on total composition weight.

In one embodiment, the composition may consist of the following:

Formula 1

| | | |
|---|---|---|
| 0.67% | by weight | Vitamin E Oil |
| 47.5755% | by weight | Extra Virgin Coconut Oil |
| 47.5755% | by weight | Aloe |
| .209% | by weight | Beta glucan |
| 3.97% | by weight | Colloidal Silver |

In another embodiment, the composition may consist of the following exemplary formula:

Formula 2

| | | |
|---|---|---|
| 1,200 | International Units | Vitamin E Oil |
| ¼ | cup | Extra Virgin Coconut Oil |
| 2 | Tablespoons | Aloe |
| 250 | Milligrams | Beta Glucan |
| 25 | Drops | Colloidal Silver |

In another embodiment, the composition may consist of the following exemplary formula:

Formula 3

| | | |
|---|---|---|
| .55%-.75% | by weight | Vitamin E Oil |
| 40%-60% | by weight | Extra Virgin Coconut Oil |
| 40%-60% | by weight | Aloe |
| 0.1%-0.3% | by weight | Beta glucan |
| 3.5%-4.5% | by weight | Colloidal Silver |

In another embodiment, the composition may consist of the following exemplary formula:

Formula 4

| | | |
|---|---|---|
| 0.6%-0.7% | by weight | Vitamin E Oil |
| 45%-55% | by weight | Extra Virgin Coconut Oil |
| 45%-55% | by weight | Aloe |
| 0.15%-0.25% | by weight | Beta glucan |
| 3.7%-4.2% | by weight | Colloidal Silver |

In another embodiment, the composition may consist of the following exemplary formula:

Formula 5

| | | |
|---|---|---|
| .66%-.68% | by weight | Vitamin E Oil |
| 46%-48% | by weight | Extra Virgin Coconut Oil |
| 46%-48% | by weight | Aloe |
| 0.19%-0.21% | by weight | Beta glucan |
| 3.8%-4.0% | by weight | Colloidal Silver |

Beeswax may be obtained from a variety of sources well known to those skilled in the art. When present in the composition, Beeswax is generally present in an amount of about 9.80% to about 18.00% by weight, more particularly from about 10.27% to about 17.77% by weight, more particularly from about 12.27% to about 17.67% by weight, more particularly from about 14.27% to about 17.57% by weight, more particularly from about 16.27% to about 17.47% by weight, and more particularly about 17.27% by weight. The beeswax may be 100% natural or organic. The addition of beeswax may improve the consistency of the composition.

Thus, in some embodiments, the composition may consist essentially of vitamin E oil, extra virgin coconut oil, aloe, beta glucan, colloidal silver, and beeswax wherein the colloidal silver is a solution of 250 parts per million colloidal silver suspended in pharmaceutical grade water and the aloe may be freeze dried aloe powder. Amounts listed in the exemplary formulas demonstrating percentages are expressed by weight based on total composition weight.

In one embodiment, the composition may consist of the following:

Formula 6

| | | |
|---|---|---|
| 8.63% | by weight | Vitamin E Oil |
| 69.06% | by weight | Extra Virgin Coconut Oil |
| 2.88% | by weight | Freeze Dried Aloe Powder |
| 0.72% | by weight | Beta glucan |
| 1.44% | by weight | Colloidal Silver |
| 17.27% | by weight | Beeswax |

In another embodiment, the composition may consist of the following exemplary formula:

Formula 7

| 1 ½ | teaspoons | Vitamin E Oil (1,300 International Units) |
|---|---|---|
| ¼ | cup | Extra Virgin Coconut Oil |
| ½ | teaspoon | Freeze Dried Aloe Powder |
| 1/8 | teaspoon | Beta Glucan (250 mg) |
| ¼ | teaspoon | Colloidal Silver (250 parts per million) |
| 1 | Tablespoon | Beeswax |

In another embodiment, the composition may consist of the following exemplary formula:

Formula 8

| | | |
|---|---|---|
| 4.00-9.50% | by weight | Vitamin E Oil |
| 60.0-79.0% | by weight | Extra Virgin Coconut Oil |
| 1.30-6.00% | by weight | Freeze Dried Aloe Powder |
| 0.25-0.90% | by weight | Beta glucan |
| 0.25-4.50% | by weight | Colloidal Silver |
| 9.80-18.00% | by weight | Beeswax |

In another embodiment, the composition may consist of the following exemplary formula:

Formula 9

| | | |
|---|---|---|
| 4.63-9.03% | by weight | Vitamin E Oil |
| 61.06-77.06% | by weight | Extra Virgin Coconut Oil |
| 2.48-5.88% | by weight | Freeze Dried Aloe Powder |
| 0.32-0.92% | by weight | Beta glucan |
| 0.44-3.44% | by weight | Colloidal Silver |
| 10.27-17.77% | by weight | Beeswax |

In another embodiment, the composition may consist of the following exemplary formula:

Formula 10

| | | |
|---|---|---|
| 5.63-8.93% | by weight | Vitamin E Oil |
| 63.06-75.06% | by weight | Extra Virgin Coconut Oil |
| 2.58-4.88% | by weight | Freeze Dried Aloe Powder |
| 0.42-0.87% | by weight | Beta glucan |
| 0.69-2.94% | by weight | Colloidal Silver |
| 12.27-17.67% | by weight | Beeswax |

In another embodiment, the composition may consist of the following exemplary formula:

Formula 11

| | | |
|---|---|---|
| 6.63-8.83% | by weight | Vitamin E Oil |
| 65.06-73.06% | by weight | Extra Virgin Coconut Oil |
| 2.68-3.88% | by weight | Freeze Dried Aloe Powder |
| 0.52-0.82% | by weight | Beta glucan |
| 0.94-2.44% | by weight | Colloidal Silver |
| 14.27-17.57% | by weight | Beeswax |

In another embodiment, the composition may consist of the following exemplary formula:

Formula 12

| | | |
|---|---|---|
| 7.63-8.73% | by weight | Vitamin E Oil |
| 67.06-71.06% | by weight | Extra Virgin Coconut Oil |
| 2.78-3.38% | by weight | Freeze Dried Aloe Powder |
| 0.62-0.77% | by weight | Beta glucan |
| 1.19-1.94% | by weight | Colloidal Silver |
| 16.27-17.47% | by weight | Beeswax |

One skilled in the art can readily determine the percentage amount of each ingredient in weight (mass) by converting each measurement to a common unit and dividing the amount of units in each individual ingredient by the total units. The presented embodiments are given as non-limiting examples and may be accurate within plus or minus 2%.

The naturopathic wound treatment may be applied topically on a skin lesion or abrasion or may be injected directly into a wound site. Those skilled in the art will appreciate that the composition may be applied on an affected area as many times as necessary to treat the wound site. For example, it is contemplated that the composition may be applied on an affected area as little as once per day, or as much as several times per day, and any amount in between as needed. Healing effects realized after application may include, but are not limited to, stimulating and regenerating healthy granulation tissue, stimulating collagen production, and pain reduction. Although anticipated use of the naturopathic wound treatment is for human skin, it is contemplated to be within the scope herein that the composition may be applied to animals as well. Generally, the ingredients may be blended in the desired proportions to form the composition and then packaged into a suitable container together with instructions for use.

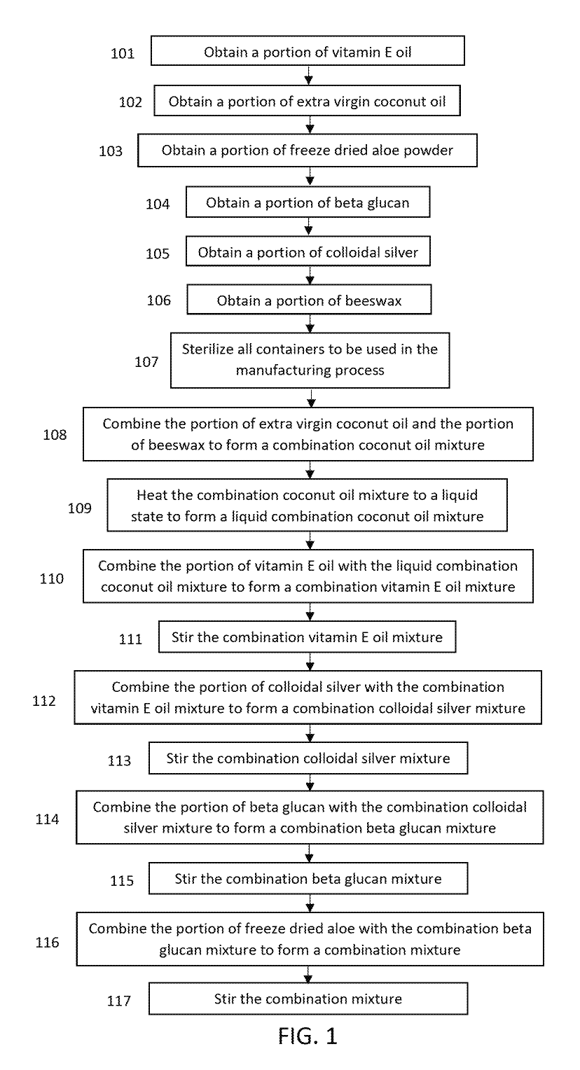

FIG. 1 depicts a method for manufacturing the naturopathic wound treatment. All ingredients may be obtained 101, 102, 103, 104, 105, 106. All containers used in manufacture of the product, including containers used for measuring, mixing, and storing, may be sterilized prior to use 107. The portion of coconut oil and beeswax may be combined in a sterile glass bowl 108. The contents of the bowl may be heated at a low temperature until the coconut oil and beeswax are in a liquid state 109. At this point, the contents may be referred to a liquid combination coconut oil mixture. The contents may be stirred before, during, or after heating. The portion of Vitamin E may be added to the liquid combination coconut oil mixture to form a combination vitamin E oil mixture 110. The combination vitamin E oil mixture may be stirred from 1 to 2 minutes 111.

The portion of colloidal silver may be added to the combination vitamin E oil mixture to form a combination colloidal silver mixture 112. The combination colloidal silver mixture may be stirred from 1 to 2 minutes 113. The portion of beta glucan may be added to the combination colloidal silver mixture to form a combination beta glucan mixture 114. The combination beta glucan mixture may be stirred from 1 to 2 minutes 115. The portion of freeze dried aloe may be added to the combination beta glucan mixture to form a combination mixture 116. The combination mixture may be stirred by hand for 5 to 10 minutes or until the desired consistency is achieved 117.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

That which is claimed is:

1. A naturopathic wound treatment product comprising a mixture of:
0.55-0.75% by weight vitamin E oil;
40-60% by weight extra virgin coconut oil;
40-60% by weight aloe;
0.1-0.3% by weight beta glucan;
3.5-4.5% by weight colloidal silver;
wherein all weights are based on a total weight of the product; and
wherein the vitamin E oil, extra virgin coconut oil, aloe, beta glucan, and colloidal silver are combined to form a single mixture.

2. The naturopathic wound treatment product of claim 1 wherein the aloe is an aloe extract.

3. The naturopathic wound treatment product of claim 1 wherein the mixture is configured to be applied onto the skin of at least one of a human and an animal.

4. The naturopathic wound treatment product according to claim 1 wherein the mixture is configured as an injectable fluid.

5. A naturopathic wound treatment product comprising:
4.0-9.5% by weight vitamin E oil;
60.0-79.0% by weight extra virgin coconut oil;
1.3-6.0% by weight freeze dried aloe powder;
0.25-0.90% by weight beta glucan;
0.25-4.50% by weight colloidal silver;
wherein all weights are based on a total weight of the product; and
wherein the vitamin E oil, extra virgin coconut oil, freeze dried aloe powder, beta glucan, and colloidal silver are combined to form a single mixture.

6. The naturopathic wound treatment product of claim 5 further comprising 9.8-18.0% by weight beeswax;
wherein the beeswax is combined with the single mixture.

7. The naturopathic wound treatment product of claim 6 wherein the vitamin E oil is 4.63-9.03% by weight, the extra virgin coconut oil is 61.06-77.06% by weight, the freeze dried aloe powder is 2.48-5.88% by weight, the beta glucan is 0.32-0.92% by weight, the colloidal silver is 0.44-3.44% by weight, and the beeswax is 10.27-17.77% by weight.

8. The naturopathic wound treatment product of claim 6 wherein the vitamin E oil is 5.63-8.93% by weight, the extra virgin coconut oil is 63.06-75.06% by weight, the freeze dried aloe powder is 2.58-4.88% by weight, the beta glucan is 0.42-0.87% by weight, the colloidal silver is 0.69-2.94% by weight, and the beeswax is 12.27-17.67% by weight.

9. The naturopathic wound treatment product of claim 6 wherein the vitamin E oil is 6.63-8.83% by weight, the extra virgin coconut oil is 65.06-73.06% by weight, the freeze dried aloe powder is 2.68-3.88% by weight, the beta glucan is 0.52-0.82% by weight, the colloidal silver is 0.94-2.44% by weight, and the beeswax is 14.27-17.57% by weight.

10. The naturopathic wound treatment product of claim 6 wherein the vitamin E oil is 7.63-8.73% by weight, the extra virgin coconut oil is 67.06-71.06% by weight, the freeze dried aloe powder is 2.78-3.38% by weight, the beta glucan is 0.62-0.77% by weight, the colloidal silver is 1.19-1.94% by weight, and the beeswax is 16.27-17.47% by weight.

11. The naturopathic wound treatment product of claim 6 wherein the vitamin E oil is 8.63% by weight, the extra virgin coconut oil is 69.06% by weight, the freeze dried aloe powder is 2.88% by weight, the beta glucan is 0.72% by weight, the colloidal silver is 1.44% by weight, and the beeswax is 17.27% by weight.

12. The naturopathic wound treatment product according to claim 5 wherein the mixture is configured as a topical ointment.

13. The naturopathic wound treatment product according to claim 5 wherein the mixture is configured as an injectable fluid.

14. The naturopathic wound treatment product according to claim 6 wherein the beeswax is 100% organic and natural.

15. A method of manufacturing a naturopathic wound treatment product that includes a portion of vitamin E oil, a portion of extra virgin coconut oil, portion of freeze dried aloe powder, a portion of beta glucan, a portion of colloidal silver, and a portion of beeswax, the method comprising the steps of:
combining the portion of extra virgin coconut oil and the portion of beeswax in a bowl to form a combination coconut oil mixture;
heating the combination coconut oil mixture to a liquid state to form a liquid combination coconut oil mixture;
combining the portion of vitamin E oil with the liquid combination coconut oil mixture to from a combination vitamin E oil mixture;
combining the portion of colloidal silver with the combination vitamin E oil mixture to form a combination colloidal silver mixture;
combining the portion of beta glucan with the combination colloidal silver mixture to form a combination beta glucan mixture;
combining the portion of freeze dried aloe with the combination beta glucan mixture to form a combination mixture;
wherein the portion of vitamin E oil is 4.0-9.5% by weight of the combination mixture;
wherein the portion of extra virgin coconut oil is 60.0-79.0% by weight of the combination mixture;
wherein the portion of freeze dried aloe powder is 1.3-6.0% by weight of the combination mixture;
wherein the portion of beta glucan is 0.25-0.90% by weight of the combination mixture;
wherein the portion of colloidal silver is 0.25-4.50% by weight of the combination mixture.

16. The method according to claim 15 wherein the portion of vitamin E oil is 4.0-9.5% by weight of the combination mixture;
wherein the portion of extra virgin coconut oil is 60.0-79.0% by weight of the combination mixture;
wherein the portion of freeze dried aloe powder is 1.3-6.0% by weight of the combination mixture;
wherein the portion of beta glucan is 0.25-0.90% by weight of the combination mixture;
wherein the portion of colloidal silver is 0.25-4.50% by weight of the combination mixture.

17. The method according to claim 15 further comprising the step of stirring the combination vitamin E oil mixture for more than one minute.

18. The method according to claim 15 further comprising the step of stirring the combination colloidal silver mixture for more than one minute.

19. The method according to claim 15 further comprising the step of stirring the combination beta glucan mixture for more than one minute.

20. The method according to claim 15 further comprising the step of stirring the combination mixture for more than five minutes.
